(19) United States
(12) Patent Application Publication
(10) Pub. No.: US 2022/0000555 A1
(43) Pub. Date: Jan. 6, 2022

(54) METHOD OF DESIGNING A SKULL PROSTHESIS, AND NAVIGATION SYSTEM

(71) Applicants: INSTITUTE OF SCIENCE AND TECHNOLOGY AUSTRIA, Klosterneuburg (AT); MEDIZINISCHE UNIVERSITÄT WIEN, Wien (AT)

(72) Inventors: Thomas AUZINGER, Gramatneusiedl (AT); Philippe DODIER, Wuernitz (AT)

(73) Assignees: INSTITUTE OF SCIENCE AND TECHNOLOGY AUSTRIA, Klosterneuburg (AT); MEDIZINISCHE UNIVERSITÄT WIEN, Wien (AT)

(21) Appl. No.: 17/280,677

(22) PCT Filed: Sep. 28, 2018
(86) PCT No.: PCT/EP2018/076526

§ 371 Date: Mar. 26, 2021

(51) Int. Cl. A61B 34/10; G06T 19/20; G06T 19/00; G16H 30/20; G16H 30/40; G16H 20/40; G16H 50/20; G16H 40/67; A61F 2/28

(52) U.S. Cl. CPC A61B 34/10; G06T 19/20; G06T 19/003; G16H 30/20; G16H 30/40; G16H 20/40; A61F 2002/4633; G16H 40/67; A61F 2/2875; G06T 2200/24; G06T 2219/2021; G06T 2210/41; G06T 2219/2004; G16H 50/20

(57) ABSTRACT

Methods and apparatus for designing a skull prosthesis are disclosed. In one arrangement, imaging data from a medical imaging process is received. The imaging data represents the shape of at least a portion of a skull. The imaging data is used to display on a display device a first virtual representation of at least a portion of the skull. User input defining a cutting line in the first virtual representation is received. A surgical operation of cutting through the skull along at least a portion of the defined cutting line to at least partially disconnect a target portion of the skull from the rest of skull is simulated. Output data is provided based on the simulation. The output data represents a simulated shape of at least a portion of the skull with the target portion at least partially disconnected from the rest of the skull, thereby defining the shape of an implantation site for a skull prosthesis to be manufactured.

The present invention relates to designing, manufacturing and implanting a skull prosthesis.

Skull prostheses (which may be referred to as implants) replace portions of the skull that have been removed during a resection operation. It is desirable in some clinical situations to perform such reconstruction immediately after the resection has been performed, as part of the same surgical operation. This is the case, for example, for resection of skull-infiltrating tumors. A common approach to performing such reconstruction is to manually shape a filling material such as polymethylmethacrylate (PMMA) or titanium mesh to form the skull prosthesis. This procedure is time consuming and can be associated with adverse events such as thermal necrosis, material fracture and infections, as well as sometimes providing unsatisfactory cosmetic results.

It is an object of the invention to improve the implantation of skull prostheses.

According to an aspect of the invention, there is provided a computer-implemented method of designing a skull prosthesis, comprising: receiving imaging data from a medical imaging process, the imaging data representing the shape of at least a portion of a skull; using the imaging data to display on a display device a first virtual representation of at least a portion of the skull; receiving user input defining a cutting line in the first virtual representation; simulating a surgical operation of cutting through the skull along at least a portion of the defined cutting line to at least partially disconnect a target portion of the skull from the rest of skull; providing output data based on the simulation, the output data representing a simulated shape of at least a portion of the skull with the target portion at least partially disconnected from the rest of the skull, thereby defining the shape of an implantation site for a skull prosthesis to be manufactured.

The method enables single-stage craniofacial reconstruction with prefabricated skull prostheses immediately after resection of skull-infiltrating pathologies. This approach represents a radical shift away from the standard single-stage approach of free-hand-molded PMMA adopted in most neurosurgical departments. As demonstrated below, large time savings are made available by avoiding the need to shape PMMA during the surgical operation. Up to 10 times faster implantation time of polyetheretherketone (PEEK) implants compared to the equally assessed PMMA reconstruction has been observed by the inventors. The time savings can desirably contribute to improving allocation of expensive surgical time. Excellent cosmesis is achieved. Complications associated with thermal necrosis and cytotoxic damage that have been reported with the traditional free-hand PMMA techniques are avoided.

The inventors have found the simulation procedure to define the shape of the skull after the target portion has been at least partially disconnected can be implemented efficiently using widely available and inexpensive consumer computing hardware, in contrast to industrial computer-aided design (CAD) based approaches for designing medical implants. Furthermore, the shape of the target portion to be removed can be fully defined using only input from a surgeon. It is not necessary to perform iterative interactions between surgeons and industrial designers to establish the shape of the target portion. The surgeon is thus able to provide all information necessary to manufacture the skull prosthesis in a single step.

In examples described below, a computed assessment of simulated and postoperative bone flap outlines shows surgical precision reflected by the mean 1.1±0.29 mm local distance between simulated and real craniotomy in cadaveric cases. No major corrections, such as recraniotomy, were needed to achieve these results. In the hypothetical situation that the surgical plan changed between the designing of the skull prosthesis and the surgical operation to implant the skull prosthesis, resulting in a modified craniotomy, the material properties of a skull prosthesis formed from a polyethylene such as PEEK or polyetherketoneketone (PEKK) would give the surgeon the ability to adapt by drilling the surplus. Due to the characteristically slow growth pattern of skull-infiltrating lesions, such as meningiomas, the scenario of over- or undersizing the skull prosthesis has to be regarded as unlikely.

In an embodiment, the output data comprises a modified version of the received imaging data, for example where a subset of voxels of the output data are modified. Provision of output data in this form can be implemented efficiently computationally. This is particularly the case where the modification comprises changing only data in the received imaging data that represents the target portion of the skull to be disconnected, which can involve modification of only a relatively small portion of the imaging data. Furthermore, this approach makes it possible for received imaging data and the output data to be provided in the same format, for example a Digital Imaging and Communications in Medicine (DICOM) format compatible with existing visualization software and neuronavigation systems.

In an embodiment, the user input further defines an angle of the simulated cutting, defined as a deviation from a normal to the surface of the skull when viewed along the cutting line, at one or more positions along the cutting line. The inventors have found that increasing the cutting angle improves stability and longevity of the implanted prosthesis, relative to perpendicular cutting through the skull.

According to an alternative aspect, there is provided a navigation system, comprising: a display device configured to display a virtual environment containing a virtual representation of at least a portion of a skull; and a reference instrument configured to indicate how a cutting line to be followed in a surgical operation can be marked, wherein: the navigation system is configured to monitor a position of the reference instrument in real space and correlate the monitored position with a position in the virtual environment, such that the position of the reference instrument relative to the skull in real space corresponds to a position of a reference instrument relative to the virtual representation of the skull in the virtual environment; and the virtual representation comprises an indication of a cutting line on the skull in the virtual environment.

The invention will now be further described, by way of example, with reference to the accompanying drawings, in which:

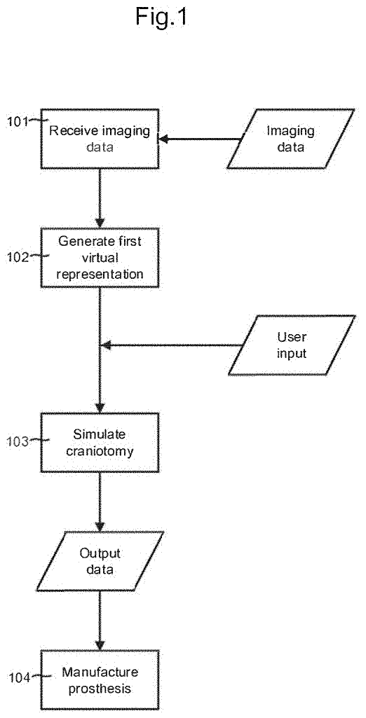

FIG. 1 is a flow chart schematically depicting a method of designing a skull prosthesis;

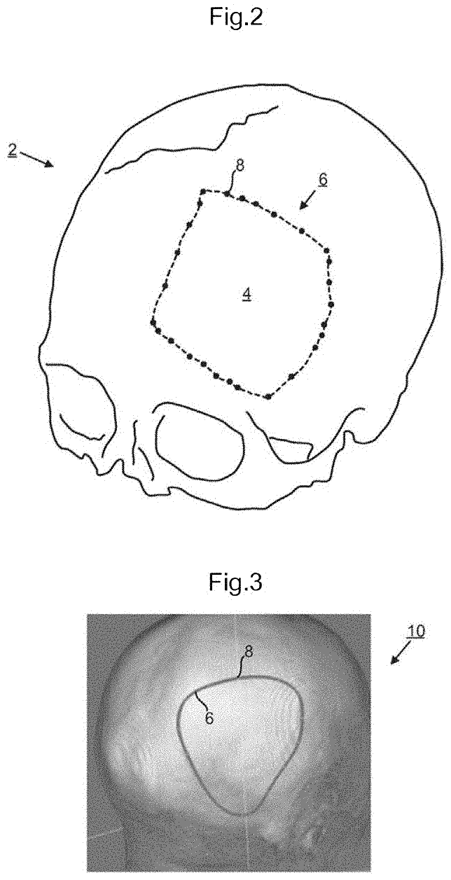

FIG. 2 is a schematic perspective view of a skull and a cutting line;

FIG. 3 is an image showing an example of a displayed first virtual representation of a skull and a cutting line defined on the surface of the skull via first reference points;

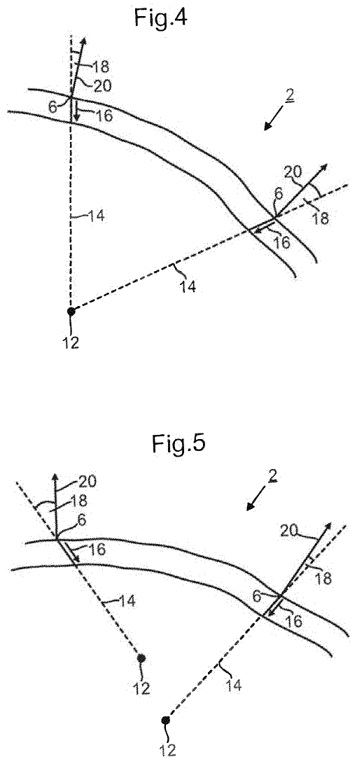

FIG. 4 is a schematic section view of a portion of a skull showing definition of angles of cutting relative to a common second reference point;

FIG. 5 is a schematic section view of a portion of a skull showing definition of angles of cutting relative to different second reference points;

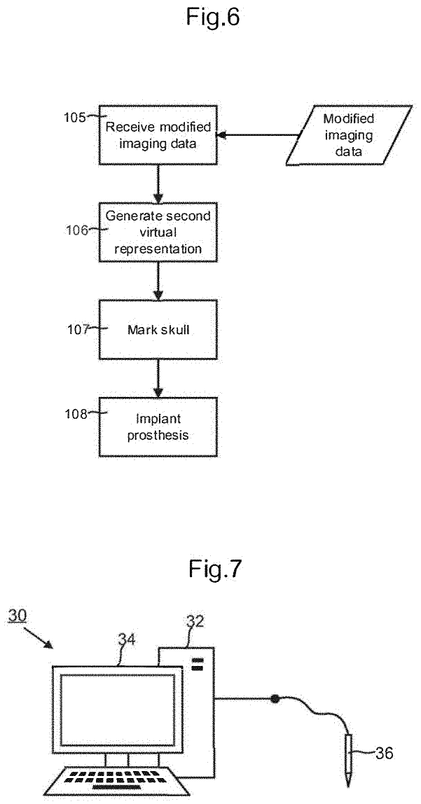

FIG. 6 is a flow chart schematically depicting a method of preparing for and implanting a skull prosthesis;

FIG. 7 schematically depicts a navigation system configured to support marking of a line on a skull that follows a cutting line used in an earlier simulation of cutting through the skull;

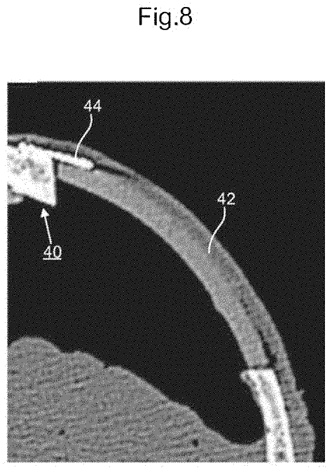

FIG. 8 is a visualisation of CT scan data of a skull after implantation of a skull prosthesis;

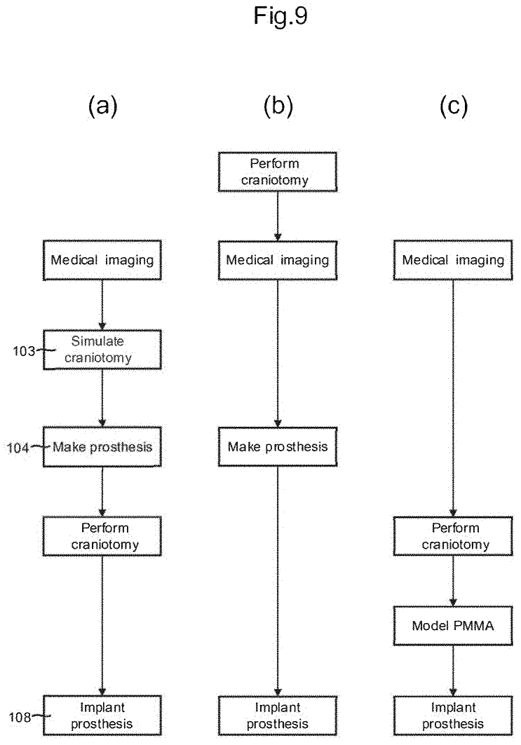

FIGS. 9(a)-(c) compare a process flow of an embodiment (FIG. 9(a)) with a process flow of a multiple stage procedure (FIG. 9(b)) and with a process flow of a free-hand-molded acrylic-based (e.g. PMMA-based) procedure (FIG. 9(c));

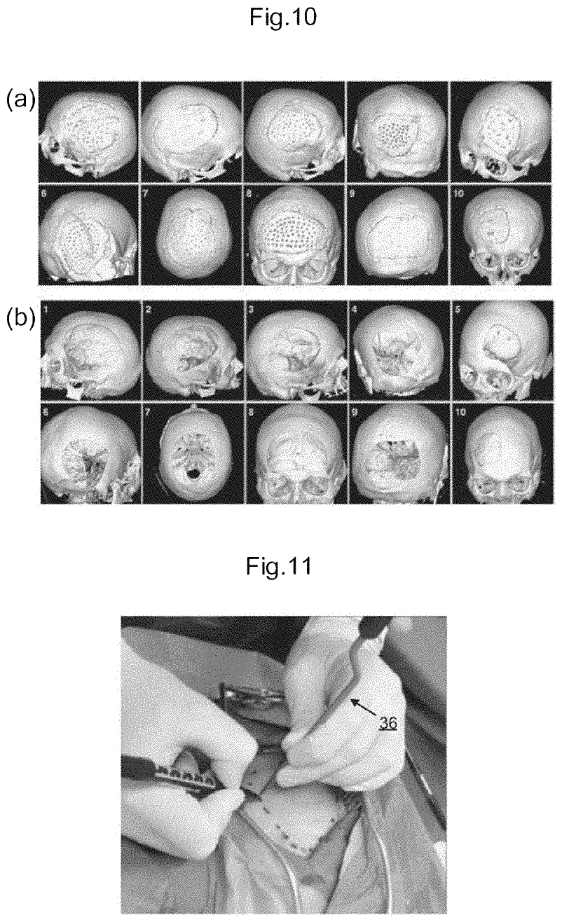

FIG. 10(a) depicts postoperative reconstructions of CT imaging data of ten clinical reference cases after resection of skull-infiltrating tumors and immediate, single-stage PMMA reconstruction;

FIG. 10(b) depicts pre-experimental virtual reconstructions of CT imaging data of 5 frozen cadaveric head specimens demonstrating the exact shape, size and location of an intended cutting away of a target portion of the skull;

FIG. 11 is a photograph showing a surgeon marking a line corresponding to a cutting line using methylene blue and a reference instrument 36 registered with a navigation system for guidance;

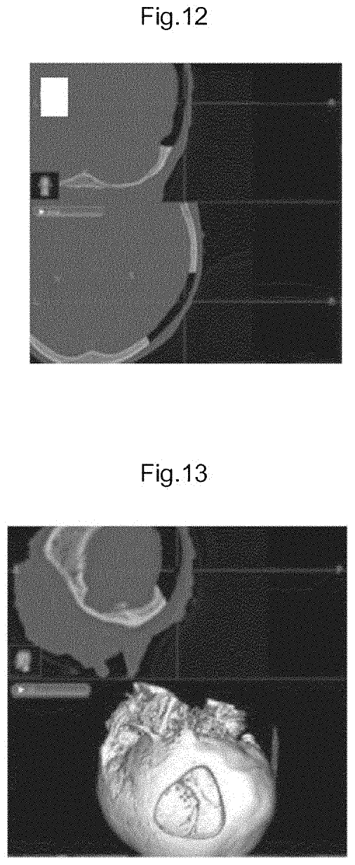

FIGS. 12 and 13 show example screen shots of a display from the navigation system during the procedure shown in FIG. 11;

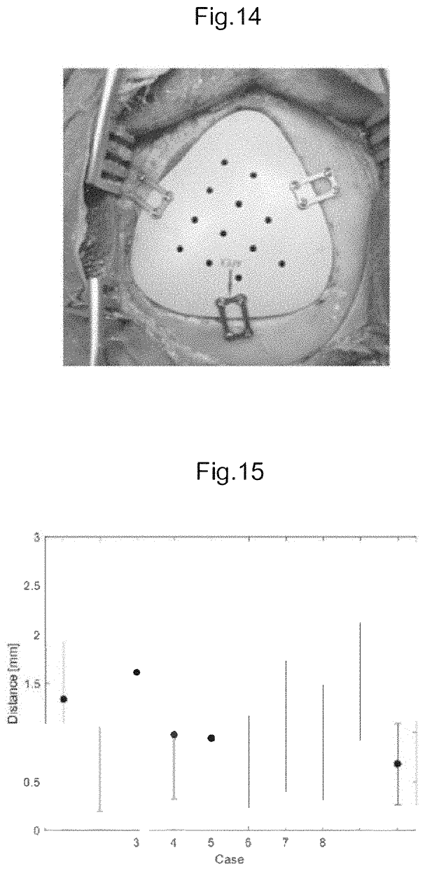

FIG. 14 is a photograph showing implantation of a skull prosthesis after a surgical operation performed based on the marking procedure illustrated in FIGS. 11-13; and

FIG. 15 is a graph showing a degree of matching between overlaid shapes of the virtual and real craniotomy evaluated by calculation of mean and standard deviation of the distances (in mm) between closest preoperative (virtual) and postoperative (actual craniotomy) points.

Various methods of the present disclosure are computer-implemented. Each step of such methods may therefore be performed by a computer. The computer may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, smart device (e.g. smart TV), etc. Alternatively, the computer may consist of a distributed computing system having plural different computers connected to each other via a network such as the internet or an intranet.

FIG. 1 is a flow chart schematically depicting a method of designing a skull prosthesis according to an embodiment. The method may be computer-implemented.

In step 101, imaging data is received. The imaging data is derived from a medical imaging process. The imaging data may be provided in the Digital Imaging and Communications in Medicine (DICOM) format. In an embodiment, the medical imaging process comprises a neurological diagnostic method, such as computed tomography (CT) scan or a magnetic resonance imaging (MRI) scan. The imaging data represents a shape of at least a portion of a skull 2 (FIG. 2), optionally prior to a surgical operation to remove a target portion 4 of the skull 2 (which may be referred to as craniotomy). The imaging data may thus comprise a plurality of voxels with each voxel containing information about the composition of material within the voxel. The relative positions of voxels identified as containing skull material identify the shape of the portion of the skull 2 being considered.

In an embodiment, the target portion 4 contains a tumor to be removed by resection.

In step 102, the imaging data is used by a computer to generate a first virtual representation of at least a portion of the skull 2 on a display device, for example a three-dimensional perspective visualization of the cranial anatomy of a portion of the skull 2. In an embodiment, the computer processes the imaging data to perform bone segmentation (i.e. to identify voxels in the imaging data that contain bone) to display the cranial anatomy. The computer may additionally process the imaging data to remove non-skull objects, such as equipment used to obtain the imaging data. The computer is programmed to allow a user to provide user input that defines a cutting line 6 (e.g. along the surface of the skull 2) in the first virtual representation. The user may, for example, interact with the displayed first virtual representation using a suitable interface (e.g. a mouse or touch sensitive interface) to define the cutting line 6. An example of a first visual representation 10 and a cutting line 6 defined by a user are shown in FIG. 3. The first virtual representation may comprise a perspective representation of the skull 2 and/or or one or more sectional representations of the skull 2.

In step 103, the surgical operation (craniotomy) of cutting through the skull 2 along the defined cutting line 6 is simulated. The simulated cutting through the skull at least partially (optionally fully) disconnects a target portion 4 of the skull 2 from the rest of the skull 2, creating an interface between the target portion 4 and the rest of the skull 2 where disconnection occurs. Output data representing a simulated shape of at least a portion of the skull 2 with the target portion 4 at least partially (optionally fully) disconnected from the rest of the skull 2 is generated (using the simulation). The output data thus defines the shape of an implantation site for a skull prosthesis to be manufactured (i.e. the gap that would be created if the target portion 4 that has been at least partially disconnected is removed from the skull 2 after being fully disconnected). Partial deconnection may comprise simulated drilling of closely spaced holes along the cutting line or simulated cutting along a large proportion of the cutting line but not all of the cutting line. In an embodiment, the simulation is used to modify the first virtual representation to represent a simulated state of the skull 2 after the simulated craniotomy and the output data is generated based on the modified first virtual representation. In an embodiment, the output data comprises a modified version of the imaging data received in step 101, which may be referred to as modified imaging data. The modification may comprise exclusively modifying a subset of the voxels of the received imaging data. In an embodiment, the modified imaging data represents a shape of the skull 2 after the simulated craniotomy. The modified imaging data thus defines the shape of the skull prosthesis to be manufactured (by defining the gap to be filled). The simulation identifies an interface surface within the thickness of the skull 2 that will be exposed by the cutting operation. The interface surface is an interface between the target portion 4 of the skull 2 when present and the rest of the skull 2. The skull prosthesis should be shaped to have an engagement surface that fits against the interface surface of the skull 2 and an outer surface that is shaped to conform with the nearby geometry of the outer surface of the skull 2 (e.g. to resemble the natural curvature of the skull 2). The skull prosthesis may be deliberately manufactured to be slightly smaller than the hole to be left by the cutting procedure (e.g. by about 1 mm around the peripheral extremity of the skull prosthesis) to ensure that the skull prosthesis can be inserted during the actual surgical operation.

In an embodiment, the generation of the modified imaging data comprises processing the received imaging data to change only data that represents the target portion 4 of the skull 2 to be disconnected by the simulated craniotomy. For example, voxels identified as containing skull material but falling within the target portion of the skull to be disconnected by the simulated craniotomy may be modified (e.g. by assigned to them voxel values corresponding to air or some other values that are different to skull tissue) so as to be distinguishable from voxels identified as containing skull material that are outside of the target portion. In an embodiment, the format of the modified imaging data output in step 103 is the same as the format of the imaging data received in step 101. For example, the modified imaging data and the received imaging data may both be in the DICOM format. This is desirable because it means the modified imaging data and the received imaging data may both be processed using standard visualization software and navigation systems such as neuronavigation systems.

In an embodiment, the user input defining the cutting line 6 is provided by the user specifying a position of each of a plurality of first reference points 8 defining the cutting line 6. This is illustrated schematically in FIG. 2 and shown also in the example first virtual representation shown in FIG. 3. Thus, it is not necessary for a user to manually define a continuous line. The cutting line can be indicated by discrete points. The positions of the first reference points 8 may be defined by point and click mouse or touch interface interactions, for example. In an embodiment, a continuous outline of the craniotomy (defining the cutting line 6) is obtained by fitting a one-dimensional curve to the first reference points 8. The curve defining the cutting line 6 may be updated after each user interaction, which allows the user to refine the planned cutting line 6 by editing, adding, and deleting points.

In an embodiment, as depicted schematically in FIGS. 4 and 5, the user input further defines an angle 18 of the simulated cutting at one or more positions along the cutting line 6. The angle 18 defines the direction 16 of cutting through the skull 2 when viewed along the cutting line 6 (e.g. into the page in the examples of FIGS. 4 and 5). The angle 18 may be defined relative to the normal (perpendicular) 20 to the surface of the skull 2. Thus, an angle 18 of zero degrees represents cutting into the skull perpendicularly. An angle 18 of 30 degrees represents cutting into the skull 2 at an angle that is 30 degrees away from perpendicular. In an embodiment, the angle 18 of the simulated cutting is defined by specifying the position of one or more second reference points 12 and requiring that, for each of one or more positions along the cutting line 6, a cutting direction 16 through the skull 2 is parallel to a line 14 extending from a selected one of the second reference points 12 to the respective position along the cutting line 6. Thus, cutting angles further away from the normal can be achieved by positioning a second reference point 12 closer to the surface of the skull 2 and cutting angles that are nearer to normal can be achieved by positioning a second reference point 12 further away from the surface of the skull 2.

In an embodiment, the angle of the simulated cutting defined by the user is greater than 10 degrees, optionally greater than 20 degrees, optionally greater than 30 degrees, optionally greater than 40 degrees, optionally greater than 50 degrees, optionally greater than 60 degrees, for at least a portion of the cutting line 6. In an embodiment, the angle of simulated cutting is defined as zero for at least a portion of the cutting line. In other embodiments, the angle of simulated cutting is defined as zero for all of the cutting line.

In an embodiment, as depicted schematically in FIG. 4, the position of the second reference point 12 is the same for all positions along the cutting line 6. In this case, the interface surface within the thickness of the skull 2 that will be exposed by the cutting operation forms part of the surface of a cone. This approach provides a desirable balance between efficiency of use and flexibility. In other embodiments, a cutting angle is made to vary (smoothly) as a function of position along the cutting line for at least a portion of the cutting line 6. An example of this type is depicted in FIG. 5, where the cutting angle is defined relative to different second reference points at different positions along the cutting line 6 (with a smooth variation of the cutting angle being achieved between the positions along the cutting line 6, optionally using interpolation or another method to defined a continual smooth variation in the cutting angle). This approach provides enhanced flexibility for adjusting the cutting angle. The inventors have found that increasing the cutting angle improves stability and longevity of the implanted prosthesis, relative to perpendicular cutting through the skull, but performing such cutting during surgery can be more difficult than perpendicular cutting. It may thus be appropriate to reduce the cutting angle near to sensitive regions and increase the cutting angle in regions where accuracy is less critical.

In step 104, the modified imaging data output by step 103 is used to manufacture the skull prosthesis. Where the modified imaging data is in the DICOM format (with a synthetically modified portion corresponding to the simulated craniotomy), for example, any of the various known techniques for manufacturing a skull prosthesis based on DICOM data may be used. In an embodiment, the skull prosthesis is manufacturing from a polyethylene such as PEEK or PEKK. The manufactured skull prosthesis may thus comprise, consist essentially of, or consist of, a polyethylene, such as PEEK or PEKK. Other example materials include acrylics such as PMMA, hydroxyapathite, silicon, ceramics, Cortoss™, and metals such as titanium.

FIG. 6 is a flow chart schematically depicting a method of preparing for and implanting a skull prosthesis.

In step 105, modified imaging data is received. The modified imaging data may be output data provided by step 103 of the method of designing a skull prosthesis described above with reference to FIG. 1. The modified imaging data may thus represent a shape of a skull 2 after a simulated craniotomy. The modified imaging data may be provided in the DICOM format.

In step 106, a second virtual representation of at least a portion of the skull is displayed using a navigation system 30. An example of a navigation system 30 is depicted schematically in FIG. 7. The navigation system 30 comprises a computer 32, a display device 34 (e.g. computer monitor), and a reference instrument 36. The navigation system 30 comprises a system of sensors allowing the navigation system 30 to monitor a position of the reference instrument 36 in real space. Such systems of sensors are well known in the art of navigation systems. In an embodiment, the reference instrument 36 comprises a needle-like tip and the navigation system 30 monitors the position of the needle-like tip. The navigation system 30 displays a virtual environment to an operator of the navigation system 30. The navigation system 30 correlates the monitored position of the reference instrument 36 in real space with a position in the virtual environment. Thus, movement of the reference instrument 36 in real space results in a corresponding movement of a representation of the reference instrument in the virtual environment. In an embodiment, the virtual environment comprises the second virtual representation of at least a portion of the skull 2. The second virtual representation is formed using the modified imaging data received in step 105, optionally merged with imaging data representing the shape of the skull prior to any simulated or real surgical operation. The navigation system 30 is configured (including calibration) so that movement of the reference instrument 36 relative to the skull 2 in the real world causes a corresponding movement of the representation of the reference instrument (or portion thereof) relative to the second virtual representation in the virtual environment. For example, movement of a tip of the reference instrument 36 over a surface of the skull in the real world causes a corresponding movement of a representation of the tip in the virtual environment over a virtual surface of the skull defined by the second virtual representation.

The navigation system 30 may be implemented by providing suitable input to any of the various neuronavigation systems known in the art (e.g. by providing DICOM data allowing generation of the second virtual representation of the skull by the neuronavigation system). Neuronavigation systems are designed to support navigation of surgical instruments within the brain during brain surgery and have not previously been used to mark a cutting line 6 on a surface of a skull for removing a target portion of the skull according a work flow of the type disclosed herein. However, the inventors have recognised that the functionality developed for brain surgery can support such work flow with minimal modification, particularly where data in a standard format such as DICOM is used.

In an embodiment, the second virtual representation is formed using the output data provided by step 103, thereby indicating the cutting line 6 to be followed. In an embodiment, the output data comprises modified imaging data that represents a shape of the skull after the simulated surgical operation has been completed. To take account of possible changes in the subject occurring between the receiving of the imaging data in step 101 and the surgical operation (which may involve delays of weeks to months), an updated version of the imaging data may be obtained, for example by repeating the medical imaging process used to provide the imaging data received in step 101 (e.g. a CT scan), a short time before the surgical operation. In such an embodiment, the second virtual representation may be provided by combining the updated version of the imaging data with the modified imaging data. Thus, updated DICOM data representing the skull just before the surgical operation may be merged with DICOM data representing the same skull after a simulated surgical operation. The resulting second virtual representation thus indicates to an operator in the displayed virtual environment where the cutting line 6 used as the basis for the simulated operation is located. The operator of the navigation system may thus mark a location of a line (e.g. as a series of dots or as a continuous line) on the skull (or on an overlay material or tissue on top of the skull) that follows (e.g. lies on top of) the cutting line 6 as indicated in the virtual environment. The marking may be performed using any of various known instruments for providing visible markings on a surface, for example by staining the surface or depositing a visible substance on the surface, for example using a marker pen or the like, by projecting a pattern of light (e.g. laser light) onto the surface, or by scratching into the surface. The marking may be performed using methylene blue for example. The second virtual representation of the skull is in registration with the skull in the real world, so the line marked on the skull will be in the same position relative to the rest of the skull as the cutting line 6 forming the basis for the simulated operation. The inventors have found that the registration and the surgical operation can be achieved with high accuracy. Thus, when a method of replacing a target portion of the skull 2 is performed in practice, including cutting through the skull 2 along the marked line provided using the above method and removing the target portion of the skull 2, the inventors have found that a subsequent step of implanting a skull prosthesis manufactured on the basis of the simulation provides an excellent fit without any manual shaping or adjustment being needed, and without new CT scan data being obtained and sent away to industrial manufacturers to provide the skull prosthesis. A methodology is thus provided which allows a skull prosthesis to be fitted during the same surgical operation as the removal of the target portion of the skull in significantly less time than is currently possible (due to the absence of manual shaping). Additionally, consistently high-quality fitting is provided which makes it easier to ensure high quality aesthetic appearance.

In the case where, prior to the simulation, user input is received defining an angle of the simulated cutting at one or more positions along the cutting line 6, an angle of the cutting may be controlled by the surgeon for each position along the marked line by reference to the user input defined angle of the simulated cutting (e.g. so as to match the user input defined angle of the simulated cutting as closely as possible).

FIG. 8 depicts a visualisation of CT scan data of a skull after implantation of a skull prosthesis according to an embodiment of the disclosure. Skull bone is labelled 40, material of the skull prosthesis is labelled 42, and a titanium clamp is labelled 44. The image demonstrates the high quality of fitting of skull prosthesis against bone that is achievable using embodiments of the present disclosure, as well as showing the advantages, in terms of improved fitting and stability, of controlling an angle of the cutting during the simulation and the cutting itself. Angling the cutting direction as described allows skull prosthesis and skull bone to overlap around the edges when viewed perpendicularly to the skull surface, which improves support of the skull prosthesis by the bone and potentially reduces mechanical load on the skull prosthesis and any associated fixation devices, which is particularly important for large skull prostheses and will in general increase long-term stability of the skull prosthesis.

FIG. 9 compares the methodology of the present disclosure (FIG. 9(a)) with alternative approaches of the prior art (FIGS. 9(b) and 9(c)) for planning cranioplastic implants to reconstruct cranial voids following resection of skull-infiltrating tumors (i.e. removal of target portions of a skull). FIG. 9(b) depicts a process flow for a multiple stage procedure, requiring a patient to undergo a primary craniotomy (top) involving resection of the skull-infiltrating tumor without reconstruction of the cranial void, and a secondary procedure (typically several weeks or months later) to implant a skull prosthesis that is manufactured based on medical imaging of the skull after the primary craniotomy. This is the standard approach for traumatic brain injuries. Relative to embodiments of the present disclosure the approach significantly increases the perioperative complication risks due to the need for two separate operations. FIG. 9(c) depicts a process flow for a single-stage procedure involving a free-hand-molded PMMA (Palacos®) reconstruction method. This approach does not require a secondary procedure to be performed at a later time but, in comparison with embodiments of the present disclosure, higher complication rates, unsatisfactory cosmetic results and longer surgical times are observed.

### Specific Examples and Experimental Validation

To validate both software and operative workflow ahead of clinical implementation, a cadaveric feasibility study was performed to assess accuracy and precision. Methods and results of this study are described below.

### Example Methods

3D volume-rendering models (3D-VR) of 43 patients treated for skull infiltrating pathologies between 2013 and 2015 by means of combined resection and large single-stage cranioplastic reconstruction (PMMA≥40 g), were reconstructed based on pre- and postoperative CT scans. Ten representative cases were selected as reference models (patient characteristics detailed in FIG. 10(a)). High-resolution CT scans were performed on five cadaveric head specimens, all provided by the Center of Anatomy and Cell Biology, Medical University of Vienna. Each specimen was matched with two clinical model cases (n=10 craniotomies), with enough distance between them to avoid overlapping.

The location of an intended craniotomy was defined by interaction with a computer-generated first virtual representation of a skull 2, as described above with reference to step 102 of FIG. 1. Automatic bone segmentation was performed to allow the cranial anatomy to be displayed. Cadaveric CT scans were used to provide the imaging data input to step 101. A planning surgeon specified positions of a plurality of first reference points to define a cutting line 6 along the outline of a desired bone flap by performing simple point-and-click mouse interactions. The continuous outline of the craniotomy was obtained by fitting a one-dimensional curve to the set of first reference points. A virtual craniotomy was created by simulating the cutting operation to remove the marked region from the skull 2. In this example, the system supported also the specification of an angle of the simulated cutting. Examples of virtual craniotomies corresponding to each of the examples of FIG. 10(a) are shown in FIG. 10(b).

The imaging data provided to step 101 and modified imaging data output from step 103 as output data were provided in the standard DICOM format. The modified imaging data thus appeared to a manufacturer of the skull prostheses as functionally identical to what would have been provided had imaging data been generated by performing a CT scan on a real skull after a real craniotomy had been performed (e.g. as in the work flow of FIG. 9(b)). The use of the DICOM format in this way allows the methodology to integrate seamlessly into current medical and industrial workflows.

The original imaging data (in DICOM format, as received in step 101 of FIG. 1) and the modified imaging data (in DICOM format, as output by step 103 in FIG. 1) were imported into a neuronavigation system (acting as navigation system 30) and merged. The neuronavigation system was a Medtronic StealthStation S7, Medtronic Surgical Technologies, Jacksonville, USA. A 3D skull model, demonstrating the delineation of the virtual craniotomy, was reconstructed to support faster marking of the craniotomy delineation with methylene blue. After fixation of the specimen in the Mayfield head holder, neuronavigation registration was performed using the combined point-merged anatomic/surface-merged registration method. The skin incision and exact craniotomy boundaries were marked under image-guidance according to the delineations of the virtual osteoclastic craniotomy. The trephination was performed according to those markings, and the prefabricated PEEK prosthesis, implanted and fixated. An example of the process is depicted in FIGS. 11-14. FIG. 11 shows a surgeon marking a line corresponding to a cutting line using methylene blue and a reference instrument 36 registered with a navigation system for guidance. FIGS. 12 and 13 show example screen shots of a display from the navigation system during the procedure shown in FIG. 11. FIG. 14 is a photograph showing implantation of a skull prosthesis after a surgical operation performed based on the marking procedure illustrated in FIGS. 11-13.

Surgical times of the work flow described above were compared with surgical times of traditional, free-hand-molded acrylic techniques in ten clinical reference cases shown in Table 1. Once the postoperative CT scans documented the PEEK implantation results, the specimens were returned to the laboratory, the PEEK alloplastics explanted and the same osteoclastic defect reconstructed with PMMA (Palacos®).

| | | | | | | | Total | |
|---|---|---|---|---|---|---|---|---|
| | | | | | | | surgical | Cadaver |
| Ref. | Sex | Age | Histology | Location | Strategy | Material | time (min) | specimen |
| 01 | f | 49 | meningothel. | convexity, | primary | PMMA | 350 | 01 |
| | | | meningioma | left | | (90 g) | | |
| | | | | frontotemporal | | | | |
| 02 | f | 47 | meningothel. | convexity, | primary | PMMA | 190 | |
| | | | meningioma | right | | (40 g) | | |
| | | | | frontoparietal | | | | |
| 03 | f | 59 | meningothel. | right | primary | PMMA | 175 | 02 |
| | | | meningioma | frontoparietal | | (50 g) | | |
| 04 | m | 54 | metastasis of | left occipital | primary | PMMA | 200 | |
| | | | esophageal | | | (40 g) | | |
| | | | carcinoma | | | | | |
| 05 | f | 55 | meningothel. | left frontal | primary | PMMA | 230 | 03 |
| | | | meningioma | | | (40 g) | | |
| 06 | f | 22 | fibrous | right | primary | PMMA | 290 | |
| | | | dysplasia | petroso- | | (120 g) | | |
| | | | | mastoideal | | | | |
| 07 | f | 59 | transitional | superior | primary | PMMA | 475 | 04 |
| | | | menigioma | sagittal | | (120 g) | | |
| | | | | sinus | | | | |
| 08 | m | 61 | esthesioneuroblastoma | bifrontal | primary and | 1. PMMA | 315 | |
| | | | | | secondary | (80 g) | | |
| | | | | | (after | 2. PEEK | | |
| | | | | | PMMA | | | |
| | | | | | infection) | | | |
| 09 | f | 73 | metastasis of | bilateral | primary | PMMA | 145 | 05 |
| | | | squamous | parieto- | | (80 g) | | |
| | | | cell | occipital | | | | |
| | | | carcinoma | | | | | |
| 10 | f | 60 | microcytic | right frontal | primary | PMMA | 180 | |
| | | | meningioma | | | (40 g) | | |

The performance of methods according to the present disclosure were evaluated through comparison of pre- and postoperative imaging data. The pre-operative imaging data was obtained using a Siemens Emotion 16 CT Scanner; scans were acquired at 130 kV (peak), mean X-ray tube current 98 mA with a field of view of 25 cm and slice thickness 0.6 mm. The post-operative imaging data was obtained using a Siemens Somatom Sensation 64 CT Scanner; scans were acquired at 120 kV, mean X-ray tube current 288 mA with a field of view of 23 cm and slice thickness 0.6 mm. Since the outline of the bone flap on the outside of the skull is the most descriptive geometrical feature of craniotomy, we compared these to determine the closeness of the match of the virtual and real craniotomy. The most relevant comparison metrics for two such outlines are their location, size, and their shape, where the former describes the accuracy of our method and the latter two its precision. A special-purpose software program was developed as a MATLAB script (MathWorks Inc., Natick, Mass., US) to compute different distance measurements between bone flap outlines. The input data was obtained by marking 3D points (ROIs) on the outline of the craniotomy in both the pre- and postoperative imaging data with the help of a conventional DICOM viewer. Operating solely on these points, the program performs several steps: (i) the points are reordered along a one-dimensional outline curve, which (ii) is fitted with a continuous curve from which (iii) a dense set of points on the outline is generated. Subsequently, (iv) the dense point sets of the pre- and postoperative data are matched to each other via a rigid transformation (i.e., a rotation and translation). Finally, (v) the closest preoperative point is determined for each postoperative point on the outline and their respective distances collected into a histogram from which the mean and standard deviation were computed. The transformation that is the output of step (iv) determines the accuracy since it describes the alignment between the virtual and real craniotomy. The precision is given by the output of step (v), since the mean and standard deviation describe how closely the shapes of the virtual and real craniotomies match when they are overlaid.

Statistical calculations included descriptive analyses (means±standard deviations). Differences between groups were evaluated by the Mann-Whitney U test and by the Wilcoxon test for paired samples. Two-sided p values below 0.05 were considered statistically significant. SPSS 23.0 software (SPSS Inc., Chicago, Ill., USA) was used for data administration and statistical calculations.

### Example Results

The shape and size of the virtual approaches corresponded to the clinical reference cases as outlined in FIGS. 10(a) and 10(b). Planning time for the delineation and segmentation of the virtual approaches (i.e. the defining of the cutting line 6 by a user) averaged 4.7±1.6 min (range 2.2-7.2 min).

The CAD-generated imaging data were compatible with the neuronavigation system used in all ten experimental cases. The point-merged anatomic/surface-merged registration and methylene blue marking of skin incisions lasted 2.2±0.7 min (range 1.3-3.2 min). The measured time for image-guided marking of the craniotomy delineation on the bone surface averaged 3.1±1.3 min (range 1.4-5.5 min). All ten prefabricated PEEK allografts were successfully implanted, with a mean implantation time of 4.2±2.1 min (range 1.4-8.5 min). Osteosynthesis of the first four implants was performed with sutures, as the titanium microfixation materials were only available at a later stage of the experiment (cases 05-10). Therefore, the more representative implantation times with microplate-osteosynthesis, as performed for the last six cases, averaged 3.0±1.2 min (range 1.4-4.5 min). These time measurements included minor adjustments to the craniotomy edges to optimize the fitting of the implants in 7 cases, with an average correction time of 1.4±0.9 min (range 0.3-2.6 min). Major corrections, such as recraniotomy, were not required.

For the sake of comparability, the same defects were reconstructed with PMMA. The implantation time of the hand-molded grafts averaged 31.1±3.8 min (range 24.4-35.8 min). Here, the most time-consuming step of the reconstruction was the PMMA polymerization phase, lasting up to 16.0 min.

Time differences became particularly apparent when both techniques were directly compared. The methodology of embodiments of the present disclosure resulted in significantly shorter reconstruction times (p=0.005, Wilcoxon test for paired samples per cranioplasty; p<0.001, overall comparison of both groups by the Mann-Whitney U test) than the traditional PMMA technique, with an average time saving of 26.8±2.3 min.

The navigational accuracy of the performed craniotomy and the surgical precision (degree of matching between the shapes of the virtual and real craniotomies when virtually overlaid) were independently evaluated. The results of the surgical precision are reflected in the mean of 1.1±0.29 mm (range 0.7-1.6 mm) of local distance between virtual and real craniotomy. Submillimetric precision was achieved in 50% of the cadaveric cases (FIG. 15).

The assessment of the global offset between virtual and actual craniotomy—as a measure of the navigational accuracy—revealed an average shift of 4.5±3.6 mm (range 1.1-13.1 mm). There were shift differences in relation to the implantation sites or head positioning. While the six cases operated upon in a “standard” supine position (cases 01/02/03/05/08/10) demonstrated more accurate results, with a mean global offset of 3.0±1.1 mm (range 1.5-4.7 mm), the four cases operated on in a prone position (cases 04/06/07/09) fared slightly worse, with a mean global offset of 6.2±5.2 mm (range 1.1-13.1 mm). Although these location-related observations did not reach statistical significance (p=0.173), the results seem to demonstrate a trend towards a reduced navigational accuracy for (sub)occipitally located implantation sites.

1. A computer-implemented method of designing a skull prosthesis, comprising:
receiving imaging data from a medical imaging process, the imaging data representing the shape of at least a portion of a skull;
using the imaging data to display on a display device a first virtual representation of at least a portion of the skull;
receiving user input defining a cutting line in the first virtual representation;
simulating a surgical operation of cutting through the skull along at least a portion of the defined cutting line to at least partially disconnect a target portion of the skull from the rest of skull;
providing output data based on the simulation, the output data representing a simulated shape of at least a portion of the skull with the target portion at least partially disconnected from the rest of the skull, thereby defining the shape of an implantation site for a skull prosthesis to be manufactured.

2. The method of claim 1, wherein the output data comprises a modified version of the received imaging data.

3. The method of claim 2, wherein the modification comprises changing only data in the received imaging data that represents the target portion of the skull to be disconnected.

4. The method of claim 3, wherein the modification comprises changing voxels of the imaging data identified as containing skull material but falling within the target portion of the skull to be distinguishable from voxels identified as containing skull material that are outside of the target portion of the skull.

5. The method of claim 1, wherein the user input defining the cutting line comprises specification of a position of each of a plurality of first reference points defining the cutting line.

6. The method of claim 1, wherein the user input further defines an angle of the simulated cutting, defined as a deviation from a normal to the surface of the skull when viewed along the cutting line, at one or more positions along the cutting line.

7. The method of claim 6, wherein the user input defines an angle of the simulated cutting, defined as a deviation from a normal to the surface of the skull when viewed along the cutting line, that varies as a function of position along the cutting line, for at least a portion of the cutting line.

8. The method of claim 6, wherein the angle of the simulated cutting is defined by specifying the position of one or more second reference points and requiring that, for each of one or more positions along the cutting line, a cutting direction through the skull is parallel to a line extending from a selected one of the second reference points to the respective position along the cutting line.

9. The method of claim 8, wherein the angle of the simulated cutting is defined relative to different second reference points for at least two different positions along the cutting line.

10. The method of claim 1, wherein the user input defining the cutting line comprises specifying a line along an outer surface of the skull.

11. The method of claim 1, wherein the medical imaging process comprises a neuroradiological diagnostic method.

12. A computer program comprising instructions which, when the program is executed by a computer, cause the computer to carry out the method of claim 1.

13. A computer-readable medium comprising instructions which, when executed by a computer, cause the computer to carry out the method of claim 1.

14. A method of manufacturing a skull prosthesis, comprising:
performing the method of claim 1; and
manufacturing a skull prosthesis using the output data.

15. The method of claim 14, wherein the skull prosthesis comprises a polyethylene.

16. A method of preparing for implantation of a skull prosthesis, comprising:
performing the method of claim 1; and
using a navigation system to mark the location of a line in real space that corresponds to the cutting line defined in the first virtual representation, wherein a position in real space of a reference instrument manipulated by a user of the navigation system is monitored and correlated with a position in a virtual environment displayed by the navigation system, wherein the virtual environment comprises a second virtual representation of at least a portion of the skull, and wherein the second virtual representation is formed using the output data representing the simulated shape of at least a portion of the skull with the target portion at least partially disconnected from the rest of the skull.

17. The method of claim 16, further comprising:
receiving an updated version of the imaging data from the medical imaging process, wherein:
the second virtual representation is generated by combining the updated version of the imaging data and the output data.

18. A method of replacing a target portion of a skull with a skull prosthesis, comprising:
performing the method of claim 14;
using a navigation system to mark the location of a line in real space that corresponds to the cutting line defined in the first virtual representation, wherein a position in real space of a reference instrument manipulated by a user of the navigation system is monitored and correlated with a position in a virtual environment displayed by the navigation system, wherein the virtual environment comprises a second virtual representation of at least a portion of the skull, and wherein the second virtual representation is formed using the output data representing the simulated shape of at least a portion of the skull with the target portion at least partially disconnected from the rest of the skull;
cutting through the skull along the marked line;
removing the target portion of the skull; and
implanting the manufactured skull prosthesis.

19. The method of claim 18, wherein:
the user input defines an angle of the simulated cutting, defined as a deviation from a normal to the surface of the skull when viewed along the cutting line, at one or more positions along the cutting line; and
during the cutting through the skull, an angle of the cutting is controlled for each position along the marked line by reference to the user input defined angle of the simulated cutting.

20. A navigation system, comprising:
a display device configured to display a virtual environment containing a virtual representation of at least a portion of a skull; and
a reference instrument configured to indicate how a cutting line to be followed in a surgical operation can be marked, wherein:
the navigation system is configured to monitor a position of the reference instrument in real space and correlate the monitored position with a position in the virtual environment, such that the position of the reference instrument relative to the skull in real space corresponds to a position of a reference instrument relative to the virtual representation of the skull in the virtual environment; and
the virtual representation comprises an indication of a cutting line on the skull in the virtual environment.
